(19) United States
(12) Patent Application Publication
(10) Pub. No.: US 2017/0296292 A1
(43) Pub. Date: Oct. 19, 2017

(54) Systems and Methods for Surgical Imaging

(71) Applicants: Eitezaz MAHMOOD, Chicago, IL (US); Faraz MAHMOOD, Brookline, MA (US); Bilal MAHMOOD, Brookline, MA (US)

(72) Inventors: Eitezaz MAHMOOD, Chicago, IL (US); Faraz MAHMOOD, Brookline, MA (US); Bilal MAHMOOD, Brookline, MA (US)

(21) Appl. No.: 15/488,234

(22) Filed: Apr. 14, 2017

Related U.S. Application Data
(60) Provisional application No. 62/323,642, filed on Apr. 16, 2016.
(60) Provisional application No. 62/352,828, filed on Jun. 21, 2016.

(51) Int. Cl. A61B 90/00; G03H 1/00; A61B 34/00; A61B 34/37; A61B 17/00; G02B 27/01; G03H 5/00; A61B 90/14; A61B 34/20; A61B 90/50

(52) U.S. Cl. CPC A61B 90/37; A61B 34/20; G03H 1/0005; A61B 34/25; A61B 34/37; A61B 17/00; G02B 27/0172; G02B 27/0179; G03H 5/00; A61B 90/14; A61B 2034/2055; A61B 2034/2065; A61B 2090/365; A61B 2090/3983; A61B 2090/502; A61B 2017/00207; G02B 2027/014; G02B 2027/0141; G02B 2027/0174; G03H 2001/0088; A61B 2017/00203

(57) ABSTRACT

Systems and methods for surgical imaging are disclosed herein. A head-mountable device (HMD) can include a display configured to provide an image within a field of view of an environment of the HIVID. At least one fiducial marker can be arranged on a surgical patient. At least one sensor can be configured to track a position of the at least one fiducial marker. Three-dimensional image information indicative of one or more internal features of the patient is provided. Based on information from the at least one sensor, a position of the surgical patient can be determined. Based on the determined position of the surgical patient, the HIVID can display at least a portion of the three-dimensional image information superimposed on at least a portion of the surgical patient within the field of view.

### RELATED DISCLOSURES

This disclosure claims priority to (i) U.S. Provisional Patent Application No. 62/323,642, titled “Systems and Methods for Surgical Imaging,” filed on Apr. 16, 2016, and (ii) U.S. Provisional Patent Application No. 62/352,828, titled “Systems and Methods for Surgical Imaging,” filed on Jun. 21, 2016, both of which are hereby incorporated by reference in their entirety.

### BACKGROUND

Medical imaging techniques allow for three-dimensional (3D) representations of various parts of the human body. For example, an X-ray computed tomography scan (CT scan) combines multiple X-ray images to produce cross-sectional images of a scanned object. Digital geometry processing can then be applied to the X-ray images to generate a 3D representation of the scanned object. Similarly, magnetic resonance imaging (MRI) can generate 3D representations by measuring a spatial distribution of water in the scanned object. Other medical imaging techniques can be used to generate 3D representations, such as ultrasound, positron emission tomography (PET), fluoroscopy, tractography, diffused tensor imaging (DTI), and nuclear magnetic resonance (NMR) spectroscopy, to name a few.

The generated 3D representation can then be observed on a display, such as a liquid crystal display (LCD) screen or the like. The 3D representation can be manipulated through rotation, resizing, slicing, etc. This process can help physicians diagnose and treat patients by allowing them to see internal features that would otherwise be hidden from view.

### SUMMARY

During surgery, it may be desirable for a surgeon to view a 3D representation of a patient's internal features superimposed on the patient in the vicinity of a surgical area. Accordingly, the systems and methods disclosed herein can provide the surgeon with an augmented reality displaying such a view.

In an aspect, a system is disclosed. The system can include a head-mountable device (HMD) with a display configured to display an image within a field of view of an environment of the HMD. The system can further include three-dimensional image information, at least one fiducial marker, and at least one sensor for tracking a position of the at least one fiducial marker. Further, the system can include a controller having a processor configured to execute instructions stored in a memory so as to perform operations. Such operations can include receiving, from the at least one sensor, information indicative of the at least one fiducial marker and, based on the received information, determining a position of a surgical patient. The operations can further include, based on the determined position of the surgical patient, displaying, via the display, at least a portion of the three-dimensional image information, where the displayed image information is superimposed on at least a portion of the surgical patient within the field of view.

In an aspect, a method is disclosed. The method can include receiving, from at least one sensor, information indicative of at least one fiducial marker and, based on the received information, determining a position of a surgical patient, where at least a portion of the surgical patient is within a field of view of an environment of a head-mountable device (HMD). The method can further include, based on the determined position of the surgical patient, displaying, via a display of the HMD, three-dimensional image information, where the three-dimensional image information is superimposed on at least a portion of the surgical patient within the field of view.

In an aspect, a system is disclosed. The system can include an HMD, where the HMD includes a display configured to display an image within a field of view of an environment of the HMD and further includes a first fiducial marker. The system can further include three-dimensional image information, a second fiducial marker, and at least one sensor for tracking positions of the first and second fiducial markers. Further, the system can include a controller having a processor configured to execute instructions stored in a memory so as to perform operations. Such operations can include receiving, from the at least one sensor, information indicative of the first and second fiducial markers and, based on the received information, determining positions of the first and second fiducial markers. The operations can further include, based on the determined positions of the first and second fiducial markers, determining positions of the HMD and a surgical patient. Further, the operations can include, based on the determined positions of the HMD and the surgical patient, displaying, via the display, at least a portion of the three-dimensional image information, where the displayed three-dimensional image information is superimposed on at least a portion of the surgical patient within the field of view.

These as well as other aspects, advantages, and alternatives, will become apparent to those of ordinary skill in the art by reading the following detailed description, with reference where appropriate to the accompanying drawings.

### BRIEF DESCRIPTION OF THE FIGURES

FIG. 1 depicts a head-mountable device (HMD) according to an example embodiment.

FIG. 2 depicts a surgical imaging system according to an example embodiment.

FIG. 3 depicts an augmented reality scenario according to an example embodiment.

FIG. 4 depicts a fiducial marker system according to an example embodiment.

FIG. 5 depicts an augmented reality scenario according to an example embodiment.

FIG. 6 is a flowchart of an example surgical imaging method according to an example embodiment.

FIG. 7 depicts a computing device according to an example embodiment.

### DETAILED DESCRIPTION

### I. Overview

A patient can be outfitted with one or more fiducial markers that are detected during medical image scanning. A surgeon can wear an HMD capable of displaying 3D image information, and the HMD can also be equipped with one or more fiducial markers. The locations of the various fiducial markers can be tracked by one or more tracking sensors. The tracking sensors can be fixed at predetermined locations within the surgical environment. Alternatively or additionally, the tracking sensors can be part of the HMD. Based on the tracked locations of the fiducial markers, a position of the patient relative to a position of the HMD can be determined.

Using data from the medical image scanning, a surgical imaging system can generate or be provided with 3D image information of one or more internal features of the patient. Based on the determined relative positions of the patient and the HMD, the HMD can display the 3D image information to the surgeon in a manner such that, from the surgeon's point of view, at least a portion of the 3D image information of the patient's internal features is superimposed on the patient and appears in the same position and orientation as the patient's actual internal features.

### II. Example Systems and Methods

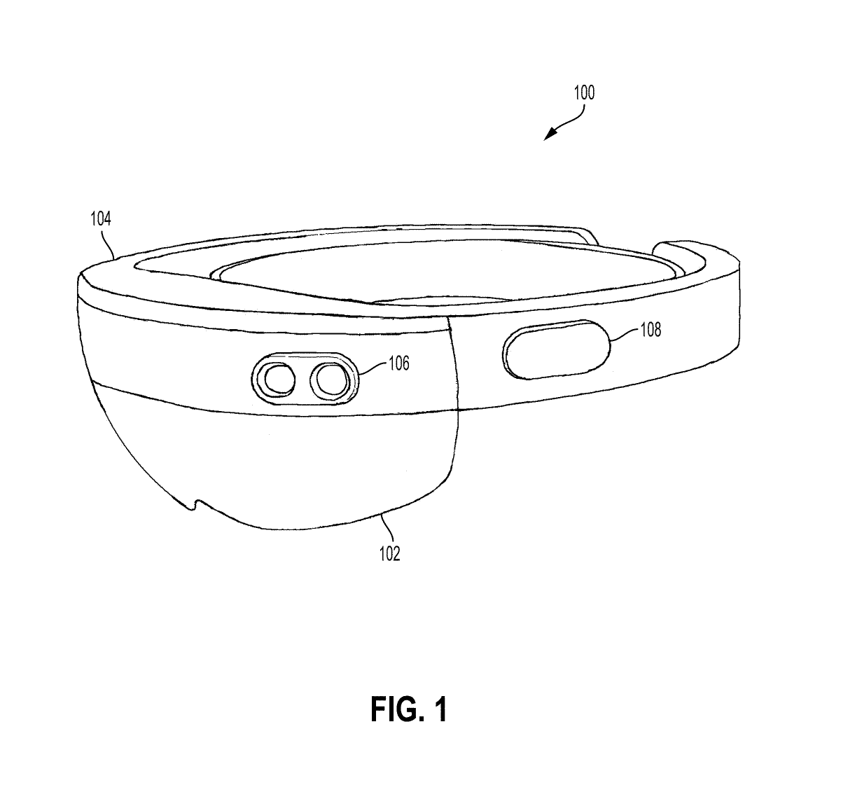

FIG. 1 depicts a head-mountable device (HMD) 100 according to an example embodiment. The HMD 100 includes a display 102, a housing 104, one or more sensors 106, and a fiducial marker 108.

The display 102 can include an electronic display screen, such as an LCD, LED, or OLED screen. Alternatively or additionally, the display 102 can include one or more transparent lenses made of glass or plastic, for instance. Such a display 102 can be configured to display, to a wearer of the HMD 100, graphical images superimposed over a real-world view. For example, where the display 102 is an electronic display screen, the sensors 106 can include a camera capable of capturing a video or image of the wearer's real-world view. The captured video or image can then be displayed on the display 102 along with one or more virtual images superimposed over the captured video or image. In examples where the display 102 includes transparent lenses, the wearer can observe the real-world view through the transparent lenses, and a projection device (not shown) can project a virtual image onto the display 102 such that the virtual image appears superimposed over the real-world view of the wearer.

The HMD 100 can display to the wearer, via the display 102, 3D image information. The 3D image information can include at least a portion of a 3D representation of a physical object, such as a 3D representation of an entire object or a planar slice of the object. The 3D image information can be displayed in various manners. For instance, the 3D information can be displayed using a holographic display, which utilizes light diffraction to create a virtual 3D image. In other examples, the 3D information can be displayed using stereoscopy in which different 2D images are displayed to the left and right eye in order to give the perception of 3D depth. Other methods of displaying the 3D image information can be used as well.

The housing 104 of the HMD 100 can include a computing system for carrying out one or more functions described herein. For instance, the housing 104 can include one or more processors configured to execute program instructions (e.g., program logic and/or machine code). The processors can include one or more general purpose processors (e.g., microprocessors) and/or one or more special purpose processors (e.g., application specific integrated circuits (ASICs) or digital signal processors (DSPs)). The housing 104 can further include memory having stored thereon the program instructions executable by the processors. The memory can take the form of a non-transitory computer-readable storage medium that can include one or more volatile and/or non-volatile storage components, such as magnetic, optical, flash, or organic storage integrated in whole or in part with the processors.

The housing 104 can further include a mounting assembly for mounting the HMD 100 on a wearer's head, where the mounting assembly includes any mechanism for securing the HMD 100 to the wearer's head. For instance, the housing 104 can include a headband configured to wrap around the circumference of the wearer's head, as shown in FIG. 1.

The fiducial marker 108 can be coupled to the housing 104. The fiducial marker 108 can be any feature capable of being detected by one or more sensors remote from the HMD 100 to determine a position of the HMD 100. For instance, the fiducial marker 108 can be retroreflective such that the marker reflects incoming light back towards a light source. Such retroreflective markers can be tracked using optical tracking systems, such as a laser tracker or a motion capture system, among others. By measuring the manner in which light is reflected off the fiducial marker 108, an optical tracking system can determine with high precision a three-dimensional location of the fiducial marker 108 relative to the optical tracking system.

Further, the fiducial marker 108 can be asymmetrical in one or more axes in order to determine an orientation of the HMD 100. For instance, as shown in FIG. 1, the fiducial marker 108 can be ovular with a major axis oriented parallel to the wearer's line of sight. By determining the orientation of the fiducial marker 108, an optical tracking system can determine the orientation of the HMD 100. Alternatively or additionally, the HMD 100 can include multiple fiducial markers in fixed positions relative to one another on the HMD 100. By determining positions of the multiple fiducial markers, the orientation of the HMD 100 can be derived.

In some examples, the fiducial marker 108 can be an electromagnetic tracking device. For instance, the fiducial marker 108 can include coils in which an electric current is induced when exposed to a time-varying magnetic field. Based on the induced electric current, a position and orientation of the fiducial marker 108 can be determined. The location and orientation of the fiducial marker 108 can be tracked using other tracking systems as well, such as directional antenna systems and acoustic systems, among others.

The HMD 100 can additionally or alternatively include an inertial measurement unit (IMU) located in the housing 104 or as part of the fiducial marker 108. The IMU can include one or more accelerometers and/or gyroscopes configured to measure various attributes of the HMD 100, such as its specific force as well as rotational attributes, such as its pitch, roll, and yaw. Based on these measurements, the computing system of the HMD 100 can determine its relative motion as well as its orientation within a three-dimensional coordinate system.

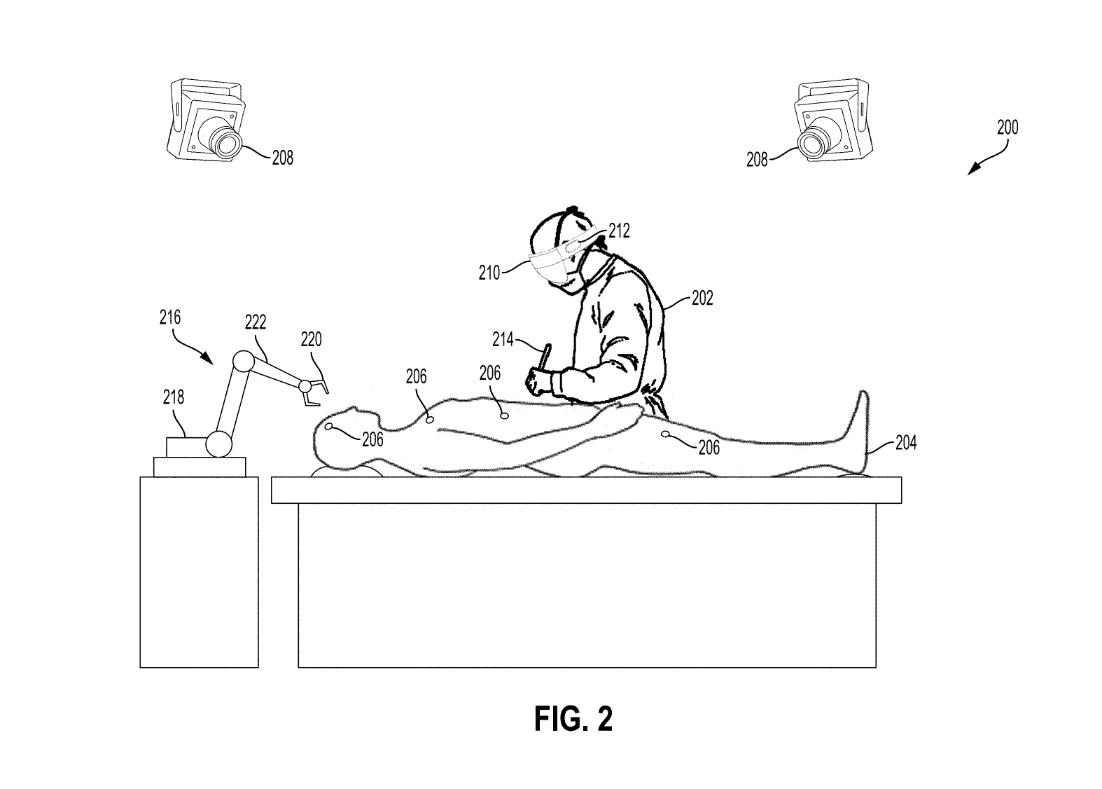

Based on the above-described features, the HMD 100 can be used within a surgical imaging system. FIG. 2 depicts a surgical imaging system 200 according to an example embodiment. Within the surgical imaging system 200, a surgeon 202 can perform a surgical operation on a patient 204. The surgeon 202 can be equipped with an HMD 210 having a fiducial marker 212. The HMD 210 and fiducial marker 212 can, for instance, be similar to the HMD 100 and fiducial marker 108 depicted in FIG. 1.

The patient 204 can be equipped with one or more fiducial markers 206. The fiducial markers 206 can be similar to the fiducial marker 108 depicted in FIG. 1. For instance, the fiducial markers 206 can be retroreflective markers tracked by an optical tracking system, electromagnetic markers tracked by an electromagnetic tracking system, etc. In some examples, the fiducial markers 206 can be arranged on a surface of a surgical drape, and the surgical drape can be draped over the patient 204.

Once the patient 204 is equipped with the fiducial markers 206, 3D image data of the patient 204 is generated. The 3D image data can be generated through a variety of techniques including, but not limited to, CT scans, MRIs, X-rays, ultrasounds, positron emission tomography (PET), fluoroscopy, tractography, diffused tensor imaging (DTI), and nuclear magnetic resonance (NMR) spectroscopy.

The fiducial markers 206 can be placed on the patient 204 such that a medical scanning procedure scans both the patient 204 and the fiducial markers 206. As a result, the 3D image data can include 3D image data of one or more of the fiducial markers 206 as well as one or more internal features of the patient 204 and can further be used to determine a position of the one or more of the fiducial markers 206 relative to the one or more internal features of the patient 204.

The surgical imaging system 200 further includes one or more tracking sensors 208. The tracking sensors 208 can determine a location of the fiducial markers 206, 212 within a 3D coordinate system. As depicted in FIG. 2, the tracking sensors 208 can be optical tracking sensors, such as motion capture cameras or laser trackers. However, in other examples, the tracking sensors 208 can include any type of tracking sensors that can track the location of the fiducial markers 206, 212 (e.g., electromagnetic trackers, directional antennas, acoustic sensors, etc.).

The tracking sensors 208 can determine a 3D position of the fiducial markers 206, 212 relative to a 3D position of the tracking sensors 208, for instance by measuring the manner in which light reflects off of the fiducial markers 206, 212. By assigning a reference point or origin, a 3D coordinate system can be established within the surgical imaging system 200. For instance, the tracking sensors 208 can be located in fixed positions in the surgical imaging system 200. The location of one of the tracking sensors 208 can be treated as the origin of the 3D coordinate system. 3D coordinates (e.g., Cartesian or polar coordinates) can then be associated with each of the fiducial markers 206, 212 based on the measured position of the fiducial markers 206, 212 relative to the tracking sensors 208.

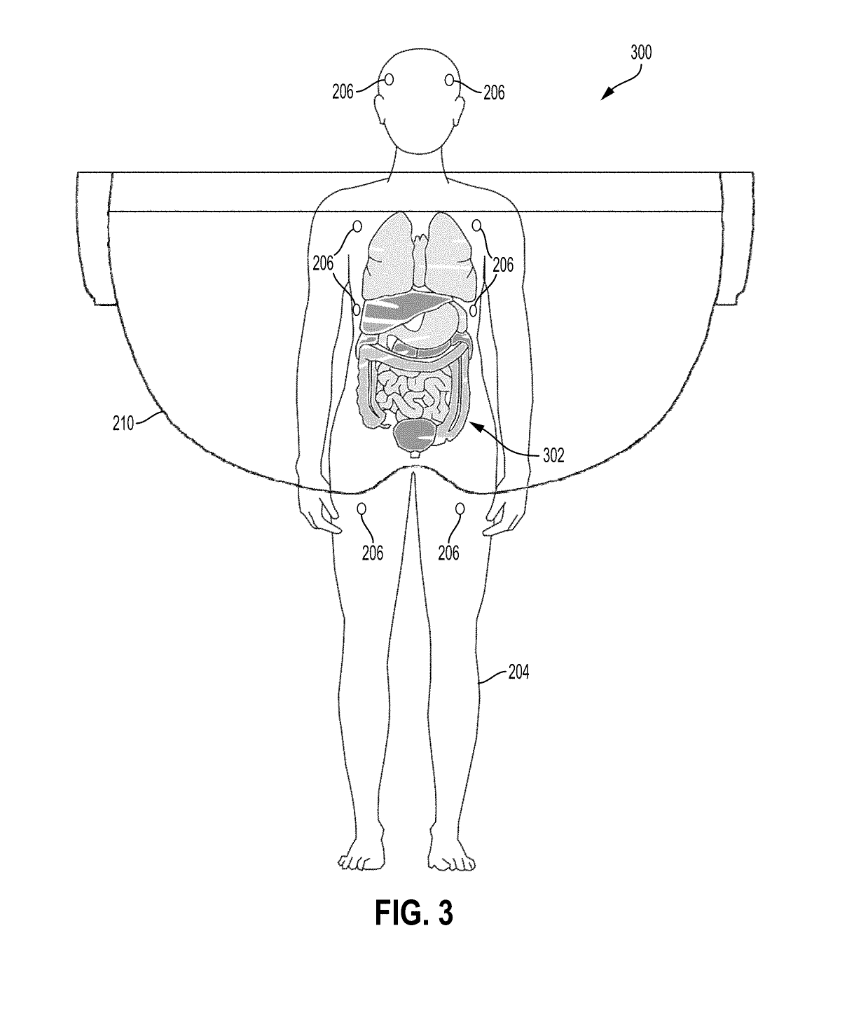

Based on the 3D coordinates of the fiducial markers 206, 212 and the determined orientation of the HMD 210, the surgical imaging system 200 can display an augmented reality within the field of view of the surgeon 202. For instance, the HMD 210 can display the captured 3D image data of one or more internal features of the patient 204 to the surgeon 202 via a display of the HMD 210. Based on the orientation of the HMD 210 and the determined relative positions of the fiducial markers 206 to the HMD 210, the HMD 210 can display the 3D image data so that the internal features of the patient 204 appears superimposed on at least a portion of the patient 204 within the field of view of the surgeon 202. Such an augmented reality scenario 300 is illustrated in FIG. 3.

The augmented reality scenario 300 illustrated in FIG. 3 depicts a field of view of the surgeon 202 through a display of the HMD 210. A 3D model 302 of internal features of the patient 204 appears superimposed on a portion of the patient 204 within the field of view. The 3D model 302 can be generated using data from a medical scan, such as a radiographic study of the patient 204.

As discussed above, the relative position of the internal features of the patient 204 to the fiducial markers 206 can be determined using data from the medical scan because the medical scan is performed after the fiducial markers are arranged on the patient 204. Further, the orientation of the HMD 210 and the position of the HMD 210 relative to the fiducial markers 206 can be determined based on position data from the tracking sensors 208. Using these determined positions and orientations, the HMD 210 can display the 3D model 302 to the surgeon 202 (or any wearer of the HMD 210) so that the 3D model 302 appears superimposed on the patient 204.

For instance, one or more data points within the 3D model 302 can correspond to a position of one or more fiducial markers 206 that were scanned during a radiographic study. Using the determined orientation and relative position of the HMD 210 to the fiducial markers 206, the HMD 210 can display the 3D model 302 to the surgeon 202 so that the positions corresponding to the one or more scanned fiducial markers 206 aligns with the actual positions of the one or more fiducial markers 206. By aligning these positions, the 3D model 302 of internal features of the patient 204 can be superimposed on the patient 204 so that they are aligned with the actual positions of the internal features.

In some instances, the 3D model 302 can depict one or more internal features of the patient 204 in particular colors based on a characteristic of the internal features. For example, based on a radiographic study of the patient 204, it can be determined that one or more internal features of the patient 204 is a tumor. Based on determining that an internal feature is a tumor, the HMD 210 can display the tumor in a particular color that is different than the colors of other internal features that are not tumors. The HMD 210 can employ such color coding based on other determined characteristics as well, including but not limited to, cancerous tissue, blood vessels, nerves, nerve pathways, etc. In some instances, the color coded internal feature can be based on an absence of internal organs in a particular area or path (e.g., a planned surgical route).

In some instances, the 3D model 302 can include features that are not representative of one or more internal features of the patient 204. For example, the 3D model 302 can alternatively or additionally include features representative of predicted post-surgery features of the patient 204. Such post-surgery features can include predicted results of cosmetic surgery (e.g., a model of the expected structure of the patient's 204 nose after undergoing rhinoplasty) as well as predicted results of non-cosmetic surgery.

Further, in addition to displaying the 3D model 302 of various internal features of the patient 204, the HMD 210 can be configured to display various vital signs (vitals) of the patient 204. Such patient vitals can include body temperature, pulse rate, respiration rate, and/or blood pressure, for instance. The patient vitals can be determined by various medical monitoring devices (e.g, a heart rate monitor, a thermometer, a respirometer, a sphygmomanometer, etc.) and communicated to the HMD 210.

Referring back to FIG. 2, the surgeon 202 can use a surgical implement 214 within the surgical imaging system 200. The surgical implement 214 can be any surgical tool used by the surgeon 202 during a surgical operation on the patient 204. It may be desirable to include in an augmented reality, such as the augmented reality scenario 300 depicted in FIG. 3, a 3D model of the surgical implement 214. Accordingly, one or more fiducial markers can be arranged on a surface of the surgical implement 214.

The tracking sensors 208 can track a position and orientation of the surgical implement 214 by tracking a position and orientation of the one or more fiducial markers on the surface of the surgical implement 214. Similar to displaying the 3D model 302 of internal features of the patient 204, the HMD 210 can display a 3D model of the surgical implement 214. For instance, based on the relative determined positions and orientations of the HMD 210 and the surgical implement 214, the HMD 210 can display the 3D model of the surgical implement 214 so that the position of the model of the surgical implement 214 relative to the 3D model 302 of the internal features is equivalent to the position of the actual surgical implement 214 relative to the actual internal features of the patient 204.

In some embodiments, the 3D model of the surgical implement 214 can be generated using predetermined 3D data (e.g., a 3D CAD model) associated with the surgical implement 214. Additionally, 3D models can be generated for various surgical implements.

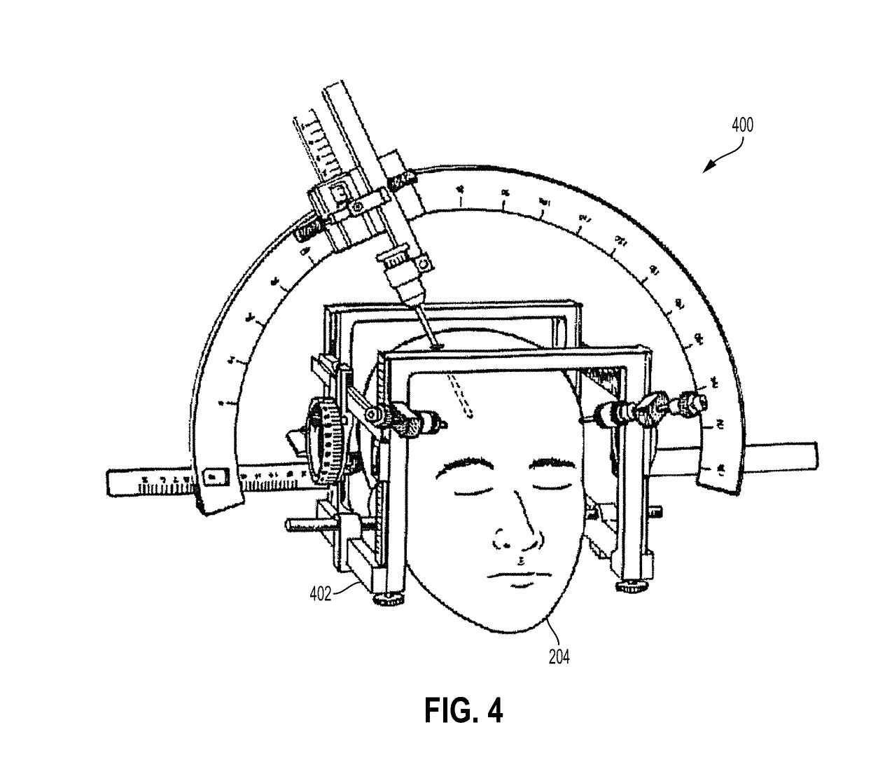

Referring next to FIG. 4, another fiducial marker system 400 is illustrated according to an example embodiment. The fiducial marker system 400 includes a stereotactic frame 402 for use in neurosurgery. The stereotactic frame 402 can include fiducial markers (not shown) that can be tracked by the tracking sensors 208. Accordingly, 3D coordinates associated with the stereotactic frame 402 within the 3D coordinate system of the surgical imaging system 200 can be determined. The relative positions of the HMD 210 and the stereotactic frame 402 can thus be determined as well.

The stereotactic frame 402 can be mounted to the head of the patient 204, and the brain of the patient 204 can be scanned (e.g., using MRI, DTI, etc.). A 3D model of the brain can be constructed based on data from the scan. For instance, the data can be used to generate a tractographic reconstruction of a neural network of the brain. Further, based on the data, a position of the brain relative to the stereotactic frame 402 can be determined. Based on the relative positions of the HMD 210, the stereotactic frame 402, and the brain, the HMD 210 can display a 3D model of the brain to the surgeon 202 so that the position of the 3D model aligns with the position of the actual brain of the patient 204.

Alternatively or additionally, the 3D model of the brain (or any other scanned internal feature of the patient 204) can be displayed to the surgeon 202 at a position that does not align with the position of the actual brain of the patient 204. For instance, the 3D model can be displayed at fixed coordinates that are offset from the coordinates of the fiducial markers 206 within the surgical imaging system 200. In some examples, the fixed coordinates can be located at a position directly above the position of the actual brain (or other internal features) of the patient 204 so that the 3D model appears above the body of the patient 204. In other examples, the fixed coordinates can be provided by one or more fiducial markers located at predetermined positions within the surgical imaging system 200.

The HMD 210 can be configured to switch between displaying the 3D model superimposed on the patient and displaying the 3D model away from the patient in response to user input. The user input can take various forms including a button press, voice input, a gesture, motion detection, etc.

While the surgical imaging system 200 depicts the patient 204 as a human patient, the patient 204 can take various forms. For instance, the patient 204 can take the form of a medical training model subjected to radiographic imaging, such as an ultrasound training model, a cardiac surgery model, a vascular surgery model, a plastic surgery model, or various other surgical training models. That is, the surgical imaging system 200 may interact with objects that may serve as stand-ins for human patients or portions thereof. In an example embodiment, such stand-in objects may include systems or devices configured to emulate or otherwise behave like the human body, e.g., imaging phantoms, artificial organs, artificial limbs, etc.

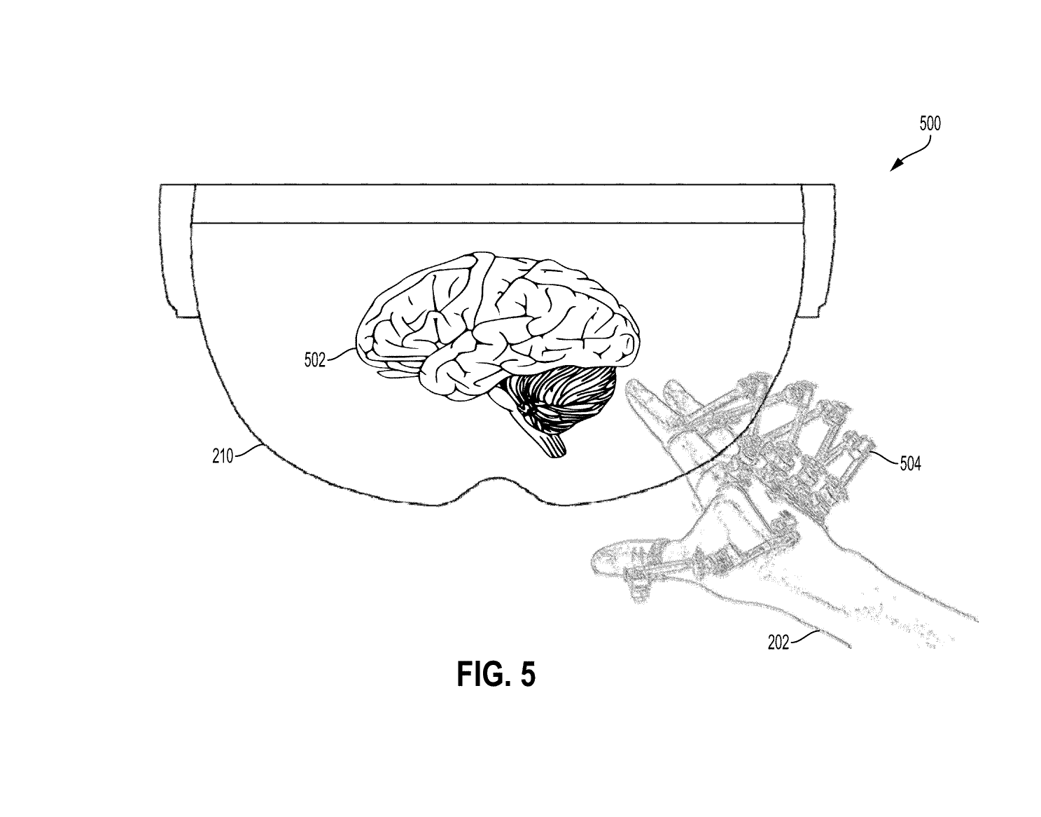

FIG. 5 illustrates an augmented reality scenario 500 in which the HMD 210 displays a 3D model 502 that is away (e.g., positionally offset) from the body of the patient 204. The 3D model 502 can be a 3D model of the brain of the patient 204, as illustrated in FIG. 5, as well as any other internal feature of the patient 204 that has been scanned and modeled in 3D.

When the 3D model 502 is displayed away from the body of the patient 204, the surgeon 202 can interact with the 3D model 502. For instance, the surgeon 202 may want to adjust a position and/or orientation of the 3D model 502 (e.g., by rotating, moving, resizing, etc.). Such adjustments can be made in response to user input, such as a button press, voice commands, a gesture, motion detection, etc.

In one example, the surgeon 202 can rotate the 3D model 502 by moving a hand from side to side. Other example gestures and responsive adjustments to the 3D model 502 are possible as well. The HMD 210 can include one or more sensors (e.g., cameras), such as the sensors 106 depicted in FIG. 1, to detect such hand movements, and the HMD 210 can adjust the 3D model 502 in response to detecting these gestures. Alternatively or additionally, the surgeon 202 can be equipped with an external device 504 that can detect various gestures from the surgeon 202 and report information associated with the various gestures to the HMD 210.

In some examples, the 3D model 502 can be displayed by the HMD 210 in such a manner as to create a field of view for the surgeon 202 as if the surgeon 202 was located inside the 3D model 502. For example, the 3D model can be enlarged such that the displayed 3D model appears several times larger than the corresponding internal features of the patient 204, and the HMD 210 can display the 3D model from a point of view located within the 3D model. The surgeon 202 can then manipulate the displayed 3D model via various gestures or commands, such as by moving the HMD 210, by using hand gestures, or issuing voice commands. For instance, the surgical imaging system 200 can detect various movements of the HMD 210 (e.g., movements caused by the surgeon 202 moving about the surgical area, tilting or turning their head, etc.), and the HMD 210 can adjust the displayed 3D model to correlate with such movements. In this manner, the HMD 210 can provide a full immersion effect as if the surgeon 202 were actually located within the internal features of the patient 204.

Further, the surgical imaging system 200 can be configured to perform robotic surgery on the patient 204 in response to interactions between the surgeon 202 and the 3D model 502. Referring back to FIG. 2, the surgical imaging system 200 can include a robotic surgical device 216. The robotic surgical device 216 can be any robotic device configured to carry out various surgical operations on the patient 204. The robotic surgical device 216 can be mounted to a reference point 218 and can include an end effector tool 220 for performing the surgical operations. The end effector tool 220 can be mounted on a robotic arm 222.

A position of the end effector tool 220 within the 3D coordinate system of the surgical imaging system 200 can be determined based on a position of the reference point 218. For instance, the reference point 218 can include a part of the robotic device 216 that is fixed in place. Accordingly, fixed 3D coordinates within the 3D coordinate system can be associated with the reference point 318. Alternatively or additionally, the reference point 318 can include a fiducial marker so that the tracking sensors 208 can track the location of the fiducial marker and determine 3D coordinates of the reference point 218.

Once the position of the reference point 218 is determined, the position of the end effector tool 220 can be determined based on an orientation of the robotic arm 222. The robotic surgery device 216 can be configured to determine the orientation of the robotic arm 222, and, based on known or otherwise determined dimensions of the robotic arm 222, the robotic surgery device 216 can be configured to determine the position of the end effector tool 220 relative to the position of the reference point 218. Using these relative positions and the 3D coordinates of the reference point 218, 3D coordinates of the end effector tool 220 can be determined. And as discussed above, 3D coordinates of one or more internal features of the patient 204 can be determined from 3D scan data that indicates the position of the internal features relative to one or more fiducial markers 206, the location of which can be determined by data from tracking sensors 208.

When the position of the end effector tool 220 and the internal features of the patient 204 are known, the robotic surgical device 216 can be configured to manipulate the end effector tool 216 to perform a surgical operation on the internal features. Such surgical operations can be carried out in response to detecting an interaction (e.g., hand gesture, voice command, movement of a stylus or surgical implement, etc.) between the surgeon 202 and a 3D model of the internal features.

The end effector tool 220 can take on various forms. In some embodiments, the end effector tool 220 can take the form of a pinching surgical tool, such as surgical forceps, needle drivers, clamps, tweezers, tongs, pliers, etc. In these cases, the end effector tool 220 can be configured to perform a pinching motion upon detecting a corresponding pinching hand gesture by the surgeon 202. For instance, the HMD 210, the external device 504, or other various sensors (e.g., positional tracking sensors located on a thumb and index finger of the surgeon 202) can detect that the surgeon 202 is performing a pinching hand gesture. Responsive to detecting the pinching hand gesture, the robotic surgery device 216 can cause the pinching surgical tool to perform a corresponding pinching motion. The robotic surgery device 216 can vary the pinching motion of the surgical tool based on the extent of the detected pinching gesture. For instance, the robotic surgery device 216 can cause the surgical tool to perform a partial pinch corresponding to a partial pinch gesture performed by the surgeon 202. In an example embodiment, a partial pinch may correspond to an end-effector tool with a pinching surgical tool that is in a partially-open configuration.

Further, based on a detected location of a hand of the surgeon 202, the surgical imaging system 200 can detect that the surgeon 202 is interacting with a 3D model of internal features of the patient 204 and responsively cause the robotic device 216 to perform one or more corresponding surgical procedures on the patient 204. For instance, the HMD 210 can include motion capture cameras to detect a location of the hand of the surgeon 202 within the 3D coordinate system. Other positional tracking sensors can be used as well, such as one or more IMUs included in the external device 504 or otherwise attached to the hand of the surgeon 202. Based on the determined location of the hand of the surgeon 202, the surgical imaging system 200 can determine the location of the hand of the surgeon 202 relative to the 3D model of internal features of the patient 204. The relative location of the hand of the surgeon 202 to the 3D model can then be used to detect an interaction between the surgeon 202 and the 3D model. For instance, the surgical imaging system 200 can detect the surgeon 202 performing a pinching gesture on one or more features of the 3D model, and the robotic surgical device 216 can responsively perform a corresponding pinching action (e.g., using forceps, needle drivers, clamps, pliers, etc.) on the corresponding actual internal feature of the patient 204.

Further based on detected interactions between the surgeon 202 and the 3D model, the surgical imaging system 200 can alter the manner in which the HMD 210 displays the 3D model to the surgeon 202. In some examples, the surgeon 202 can “draw” on the 3D model by performing one or more gestures on the 3D model, and the HMD 210 can superimpose 3D image data onto the 3D model that corresponds to the gestures. For instance, the surgeon 202 can make a hand gesture to draw a surgical path by interacting with a 3D model that is displayed away from the patient 204, and the HMD 210 can then display the drawn surgical path superimposed on the patient 204.

In some embodiments, in response to the surgical path being superimposed on the patient, various surgical outcomes may be predicted and presented to the surgeon 202 via the HMD 210. For example, the drawn surgical path may result in a given predicted bleeding rate. Such a predicted bleeding rate may be provided to the surgeon 202 via the display of HMD 210 or via other means, such as an audio alert. As another example, the drawn surgical path may result in a given predicted tumor excision likelihood. Other types of predicted information may be presented to the surgeon 202 based on the drawn surgical path as well.

In some examples, in addition to or as an alternative to the tracking sensors 208, the surgical imaging system 200 can use one or more sensors on the HMD 210, such as the sensors 106 depicted in FIG. 1, to determine the position of various objects and devices within the surgery system 200. For instance, the HMD 210 can include a camera for detecting a position of the fiducial markers 206 relative to a position of the HMD 210. Further, the camera can be used to detect various user input gestures for interacting with a 3D model.

In other examples, the surgical imaging system 200 can include one or more additional HMDs. For instance, another person assisting or observing the surgeon 202 can be equipped with an additional HMD. These additional HMDs can similarly be equipped with sensors to determine the position of various objects and devices within the surgical imaging system 200. Further, the additional HMDs can similarly display to their wearer a 3D model of internal features of the patient 204. The 3D model can appear superimposed on the patient 204 or away from the body of the patient 204. Alternatively, the display of the HMD 210 of the surgeon 202 can be replicated and displayed on displays of the additional HMDs.

In some examples, the surgical imaging system 200 can further include one or more cameras located inside the patient 204. Such internal cameras can be used for determining a position of one or more objects located inside the patient 204 (e.g., for determining a position of the surgical implement 214 during surgery). For example, during laparoscopic surgery, a telescopic camera can be inserted into the abdomen of the patient 204. Other examples are possible as well. Further, in some examples, a video feed from these internal cameras can be transmitted to the HMD 210 and displayed to the surgeon 202. The surgeon 202 can also control one or more movements of these cameras via the HMD 210. For instance, the HMD 210 can detect movement (e.g., around the x-, y-, and z-axes) of the head of the surgeon 202, and an orientation of an internal camera can be adjusted to match the detected movement. That is, the surgeon 202 may be able to control an orientation of the internal camera (and a corresponding perspective of the displayed video feed) based on an orientation of the HMD 210.

In other examples, the position and arrangement of the 3D model can be updated in real time. For instance, the patient 204 can be exposed to real time radiographic imaging, during which one or more internal features of the patient 204 are repeatedly radiographically scanned. After each scan, an updated 3D model of the features can be generated and displayed by the HMD 210.

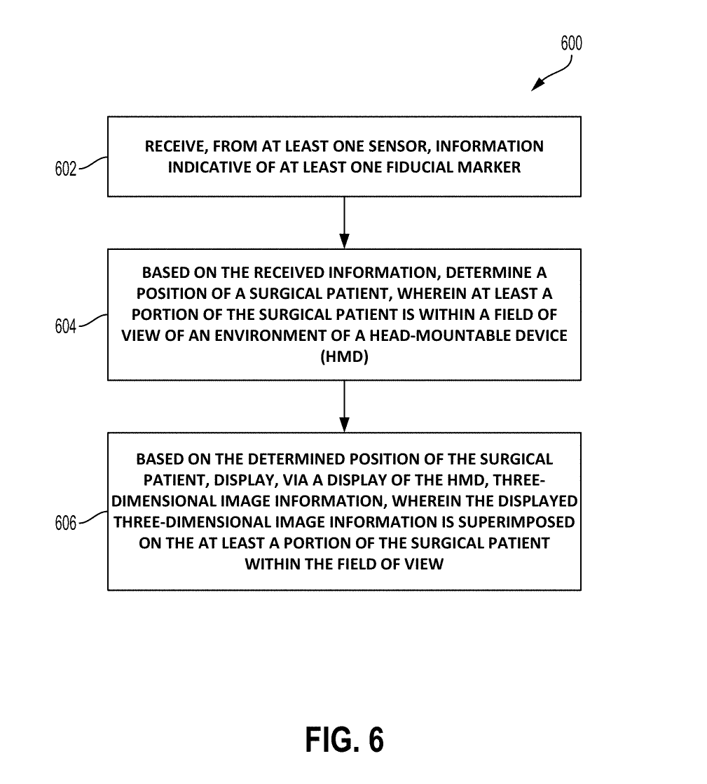

Referring next to FIG. 6, a flowchart is shown of an example surgical imaging method 600 according to an example embodiment. The example method 600 can include one or more operations, functions, or actions, as depicted by one or more of blocks 602, 604, and/or 606, each of which can be carried out by any of the systems described by way of FIGS. 1-5; however, other configurations could be used as well.

Furthermore, those skilled in the art will understand that the flowchart described herein illustrates functionality and operation of certain implementations of example embodiments. In this regard, each block of the flowchart can represent a module, a segment, or a portion of program code, which includes one or more instructions executable by a processor for implementing specific logical functions or steps in the process. The program code can be stored on any type of computer readable medium, for example, such as a storage device including a disk or hard drive. In addition, each block can represent circuitry that is wired to perform the specific logical functions in the process. Alternative implementations are included within the scope of the example embodiments of the present application in which functions can be executed out of order from that shown or discussed, including substantially concurrent or in reverse order, depending on the functionality involved, as would be understood by those reasonably skilled in the art.

Method 600 begins at block 602, which includes receiving, from at least one sensor, information indicative of at least one fiducial marker. The at least one sensor can include one or more tracking sensors (e.g., optical tracking sensors, acoustic tracking sensors, directional antennas, etc.). The tracking sensors can be positioned at fixed locations throughout a 3D coordinate system and/or can include sensors mounted on one or more HMDs. The information received from the at least one sensor can include a position of the at least one fiducial marker relative to a position of the at least one sensor.

Method 600 continues at block 604, which includes, based on the received information, determining a position of a surgical patient, wherein at least a portion of the surgical patient is within a field of view of an environment of an HMD. The position of the surgical patient can be determined based on the position of the at least one fiducial marker, which can be arranged on the surgical patient. The position of the at least one fiducial marker can be determined based on known or otherwise determined positions of the one or more tracking sensors and the relative position of the fiducial marker to the tracking sensors.

Method 600 continues at block 606, which includes, based on the determined position of the surgical patient, displaying, via a display of the HMD, three-dimensional image information, wherein the displayed three-dimensional image information is superimposed on the at least a portion of the surgical patient within the field of view. The three-dimensional image information can include a 3D model of one or more internal features of the patient based on a radiographic study of the patient.

In addition to the operations depicted in FIG. 6, other operations can be utilized with the example surgical imaging systems presented herein.

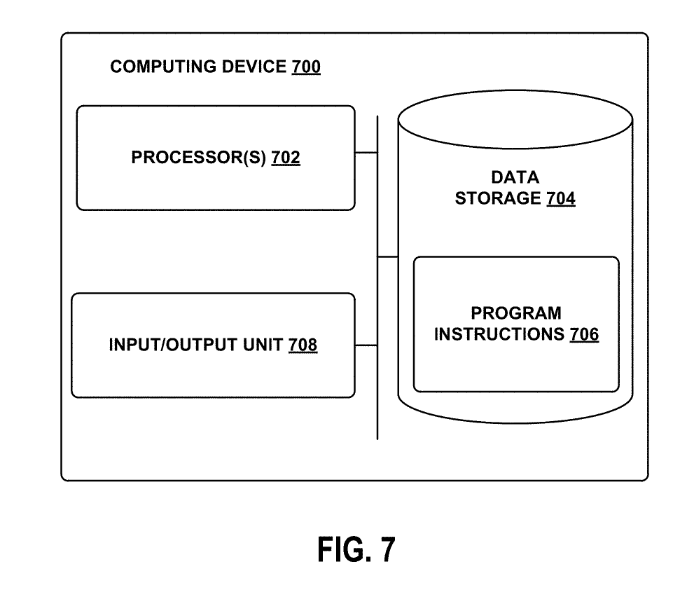

In order to carry out the methods, processes, or functions disclosed herein, the surgical imaging system 200 can include various computing device components. FIG. 7 illustrates a computing device 700 according to an example embodiment.

The computing device 700 can include one or more processors 702, data storage 704, program instructions 706, and an input/output unit 708, all of which can be coupled by a system bus or a similar mechanism. The one or more processors 702 can include one or more central processing units (CPUs), such as one or more general purpose processors and/or one or more dedicated processors (e.g., application specific integrated circuits (ASICs) or digital signal processors (DSPs), etc.). The one or more processors 702 can be configured to execute computer-readable program instructions 706 that are stored in the data storage 704 and are executable to provide at least part of the functionality described herein.

The data storage 704 can include or take the form of one or more computer-readable storage media that can be read or accessed by at least one of the one or more processors 702. The one or more computer-readable storage media can include volatile and/or non-volatile storage components, such as optical, magnetic, organic, or other memory or disc storage, which can be integrated in whole or in part with at least one of the one or more processors 702. In some embodiments, the data storage 704 can be implemented using a single physical device (e.g., one optical, magnetic, organic, or other memory or disc storage unit), while in other embodiments, the data storage 704 can be implemented using two or more physical devices.

The input/output unit 708 can include user input/output devices, network input/output devices, and/or other types of input/output devices. For example, input/output unit 708 can include user input/output devices, such as a touch screen, a keyboard, a keypad, a computer mouse, liquid crystal displays (LCD), light emitting diodes (LEDs), displays using digital light processing (DLP) technology, cathode ray tubes (CRT), light bulbs, and/or other similar devices. Network input/output devices can include wired network receivers and/or transceivers, such as an Ethernet transceiver, a Universal Serial Bus (USB) transceiver, or similar transceiver configurable to communicate via a twisted pair wire, a coaxial cable, a fiber-optic link, or a similar physical connection to a wireline network, and/or wireless network receivers and/or transceivers, such as a Bluetooth transceiver, a Zigbee transceiver, a Wi-Fi transceiver, a WiMAX transceiver, a wireless wide-area network (WWAN) transceiver and/or other similar types of wireless transceivers configurable to communicate via a wireless network.

The computing device 700 can be implemented in whole or in part in various components of the surgical imaging system 200. For instance, the computing device 700 can be implemented in whole or in part in the HMD 210 and/or in at least one device remotely located from the HMD 210, such as a workstation or personal computer. Generally, the manner in which the computing device 700 is implemented can vary, depending upon the particular application.

### III. Example Embodiments

In one aspect, a system can include a head-mountable device (HMD), wherein the HMD comprises a display configured to provide an image within a field of view of an environment of the HMD; at least one fiducial marker; at least one sensor for tracking a position of the at least one fiducial marker; three-dimensional image information; and a controller, wherein the controller includes a processor configured to execute instructions stored in a memory so as to perform operations, the operations comprising receiving, from the at least one sensor, information indicative of the at least one fiducial marker; based on the received information, determining a position of a surgical patient; and based on the determined position of the surgical patient, displaying, via the display, at least a portion of the three-dimensional image information, wherein the displayed image information is superimposed on at least a portion of the surgical patient within the field of view.

In some embodiments, the system can further include a surgical drape, wherein the at least one fiducial marker is arranged on at least one surface of the surgical drape.

In some embodiments, the system can further include at least one surgical implement, wherein the at least one fiducial marker is arranged on at least one surface of the at least one surgical implement.

In some embodiments of the system, the three-dimensional image information can include information based on at least one radiographic study of the surgical patient.

In some embodiments of the system, the three-dimensional image information can include information based on at least one tractographic reconstruction of a neural network of a brain of the surgical patient.

In some embodiments of the system, the three-dimensional image information can include information based on real-time radiographic imaging of the surgical patient.

In some embodiments of the system, the three-dimensional image information can include a holographic model of at least a portion of the surgical patient.

In some embodiments, the system can further include a robotic surgical device, wherein the operations executed by the processor further comprise receiving information indicative of a gesture, wherein the gesture comprises a control input for the robotic surgical device; and responsive to receiving the information indicative of the gesture, causing the robotic surgical device to perform a surgical act.

In some embodiments, the system can further include at least one other HMD, wherein determining the position of the surgical patient is further based on a position of the at least one other HMD.

In some embodiments, the system can further include at least one other HMD, wherein determining the position of the surgical patient is further based on a position of the at least one other HMD.

In a further aspect, a method can include receiving, from at least one sensor, information indicative of at least one fiducial marker; based on the received information, determining a position of a surgical patient, wherein at least a portion of the surgical patient is within a field of view of an environment of a head-mountable device (HMD); and based on the determined position of the surgical patient, displaying, via a display of the HMD, three-dimensional image information, wherein the displayed three-dimensional image information is superimposed on the at least a portion of the surgical patient within the field of view.

In some embodiments of the method, the at least one fiducial marker can be arranged on at least one surface of a surgical drape.

In some embodiments of the method, the at least one fiducial marker can be arranged on at least one surface of at least one surgical implement.

In some embodiments of the method, the three-dimensional image information can include information based on at least one radiographic study of the surgical patient.

In some embodiments of the method, the three-dimensional image information can include information based on at least one tractographic reconstruction of a neural network of a brain of the surgical patient.

In some embodiments of the method, the three-dimensional image information can include information based on real-time radiographic imaging of the surgical patient.

In some embodiments of the method, the three-dimensional image information can include a holographic model of at least a portion of the surgical patient.

In some embodiments, the method can further include receiving information indicative of a gesture, wherein the gesture comprises a control input for a robotic surgical device; and responsive to receiving information indicative of the gesture, causing the robotic surgical device to perform a surgical act.

In some embodiments, the method can further include providing at least one other HMD, wherein determining the position of the surgical patient is further based on a position of the at least one other HMD.

In yet a further aspect, a system can include a head-mountable device (HMD), wherein the HMD comprises a display configured to provide an image within a field of view of an environment of the HMD, and wherein the HMD further comprises a first fiducial marker; a second fiducial marker; at least one sensor for tracking positions of the first and second fiducial markers; three-dimensional image information; and a controller, wherein the controller includes a processor configured to execute instructions stored in a memory so as to perform operations, the operations comprising receiving, from the at least one sensor, information indicative of the first and second fiducial markers; based on the received information, determining positions of the first and second fiducial markers; based on the determined positions of the first and second fiducial markers, determining positions of the HMD and a surgical patient; and based on the determined positions of the HMD and the surgical patient, displaying, via the display, at least a portion of the three-dimensional image information, wherein the displayed three-dimensional image information is superimposed on at least a portion of the surgical patient within the field of view.

### IV. CONCLUSION

The particular arrangements shown in the Figures should not be viewed as limiting. It should be understood that other embodiments can include more or less of each element shown in a given Figure. Further, some of the illustrated elements can be combined or omitted. Yet further, an exemplary embodiment can include elements that are not illustrated in the Figures.

Additionally, while various aspects and embodiments have been disclosed herein, other aspects and embodiments will be apparent to those skilled in the art. The various aspects and embodiments disclosed herein are for purposes of illustration and are not intended to be limiting, with the true scope being indicated by the claims. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are contemplated herein.

What is claimed is:

1. A system comprising:
a head-mountable device (HMD), wherein the HMD comprises a display configured to provide an image within a field of view of an environment of the HMD;
at least one fiducial marker;
at least one sensor for tracking a position of the at least one fiducial marker;
three-dimensional image information; and
a controller, wherein the controller comprises a processor configured to execute instructions stored in a memory so as to perform operations, the operations comprising:
receiving, from the at least one sensor, information indicative of the at least one fiducial marker;
based on the received information, determining a position of a surgical patient; and
based on the determined position of the surgical patient, displaying, via the display, at least a portion of the three-dimensional image information, wherein the displayed image information is superimposed on at least a portion of the surgical patient within the field of view.

2. The system of claim 1, further comprising a surgical drape, wherein the at least one fiducial marker is arranged on at least one surface of the surgical drape.

3. The system of claim 1, further comprising at least one surgical implement, wherein the at least one fiducial marker is arranged on at least one surface of the at least one surgical implement.

4. The system of claim 1, wherein the three-dimensional image information comprises information based on at least one radiographic study of the surgical patient.

5. The system of claim 1, wherein the three-dimensional image information comprises information based on at least one tractographic reconstruction of a neural network of a brain of the surgical patient.

6. The system of claim 1, wherein the three-dimensional image information comprises information based on real-time radiographic imaging of the surgical patient.

7. The system of claim 1, wherein the three-dimensional image information comprises a holographic model of at least a portion of the surgical patient.

8. The system of claim 1, further comprising a robotic surgical device, wherein the operations further comprise:
receiving information indicative of a gesture, wherein the gesture comprises a control input for the robotic surgical device; and
responsive to receiving the information indicative of the gesture, causing the robotic surgical device to perform a surgical act.

9. The system of claim 1, further comprising at least one other HMD, wherein determining the position of the surgical patient is further based on a position of the at least one other HMD.

10. The system of claim 1, further comprising a second display, and wherein the operations further comprise displaying, via the second display, at least a portion of the three-dimensional image information.

11. A method comprising:
receiving, from at least one sensor, information indicative of at least one fiducial marker;
based on the received information, determining a position of a surgical patient, wherein at least a portion of the surgical patient is within a field of view of an environment of a head-mountable device (HMD); and
based on the determined position of the surgical patient, displaying, via a display of the HMD, three-dimensional image information, wherein the displayed three-dimensional image information is superimposed on the at least a portion of the surgical patient within the field of view.

12. The method of claim 11, wherein the at least one fiducial marker is arranged on at least one surface of a surgical drape.

13. The method of claim 11, wherein the at least one fiducial marker is arranged on at least one surface of at least one surgical implement.

14. The method of claim 11, wherein the three-dimensional image information comprises information based on at least one radiographic study of the surgical patient.

15. The method of claim 11, wherein the three-dimensional image information comprises information based on at least one tractographic reconstruction of a neural network of a brain of the surgical patient.

16. The method of claim 11, wherein the three-dimensional image information comprises information based on real-time radiographic imaging of the surgical patient.

17. The method of claim 11, wherein the three-dimensional image information comprises a holographic model of at least a portion of the surgical patient.

18. The method of claim 11, further comprising:
receiving information indicative of a gesture, wherein the gesture comprises a control input for a robotic surgical device; and
responsive to receiving information indicative of the gesture, causing the robotic surgical device to perform a surgical act.

19. The method of claim 11, further comprising providing at least one other HMD, wherein determining the position of the surgical patient is further based on a position of the at least one other HMD.

20. A system comprising:
a head-mountable device (HMD), wherein the HMD comprises a display configured to provide an image within a field of view of an environment of the HMD, and wherein the HMD further comprises a first fiducial marker;
a second fiducial marker;
at least one sensor for tracking positions of the first and second fiducial markers;
three-dimensional image information; and
a controller, wherein the controller comprises a processor configured to execute instructions stored in a memory so as to perform operations, the operations comprising:
receiving, from the at least one sensor, information indicative of the first and second fiducial markers;
based on the received information, determining positions of the first and second fiducial markers;
based on the determined positions of the first and second fiducial markers, determining positions of the HMD and a surgical patient; and
based on the determined positions of the HMD and the surgical patient, displaying, via the display, at least a portion of the three-dimensional image information, wherein the displayed three-dimensional image information is superimposed on at least a portion of the surgical patient within the field of view.
